(19) United States
(12) Patent Application Publication
(10) Pub. No.: US 2010/0121195 A1
(43) Pub. Date: May 13, 2010

(54) MEDICAL INSTRUMENT

(76) Inventor: Hak Il Kang, Seoul (KR)

Correspondence Address: JONES DAY, 222 EAST 41ST ST, NEW YORK, NY, 10017, US

(21) Appl. No.: 12/618,174

(22) Filed: Nov. 13, 2009

(30) Foreign Application Priority Data
Nov. 13, 2008 (KR) 10-2008-0112664

(51) Int. Cl. A61B 8/14

(52) U.S. Cl. 600/459

(57) ABSTRACT

Provided is a medical instrument that may include a probe connector on a top surface of an extended board in order to provide a diagnostic probe capable of performing ultrasonic diagnosis in a body. The extended board is externally extended from a main board that is included in the body. By forming the probe connector on the top surface of the extended board, it is possible to connect the diagnostic probe and the body without using a separate device. Accordingly, it is possible to simplify a configuration of mounting the diagnostic probe to the body and to reduce a time of mounting the diagnostic probe to the body.

### CROSS-REFERENCE TO RELATED APPLICATION

This application claims the benefit of Korean Patent Application No. 10-2008-0112664, filed on Nov. 13, 2008, in the Korean Intellectual Property Office, the disclosure of which is incorporated herein by reference.

### BACKGROUND

1. Field of the Invention

The present invention relates to a medical instrument, and more particularly, to a medical instrument that may provide a probe connector on a top surface of the medical instrument to make it possible to readily mount a diagnostic probe to the medical instrument.

2. Description of the Related Art

A medical instrument may be used to inspect an inspection subject. In particular, an ultrasonic medical instrument that inspects a portion of a body of the inspection subject may use a diagnostic probe to generate ultrasonic waves.

An ultrasonic diagnostic apparatus may include a diagnostic probe to transmit and receive ultrasonic waves, and a body including a main board to control transmission/reception of the ultrasonic waves of the diagnostic probe and to process received signals. Here, the body may also include a display unit to display, using an image, information that is transmitted and received via the diagnostic probe. Also, the body may further include a control panel to input an inspection content of the inspection subject or other information.

Upon a user's request, the medical instrument may be provided in a fixed space or a particular space. In particular, when the inspection subject is not readily movable, the medical instrument may need to be moved. Along with the movement of the medical instrument, the diagnostic probe may also need to be moved. After an auxiliary plate is attached to a side of the body, the diagnostic probe may be mounted to the body.

Specifically, in order to use the diagnostic probe in the movable medical instrument, a probe connector may be required to connect the body and the diagnostic probe. Generally, the probe connector is provided on the side of the body whereby a probe is connected in a side direction. In this instance, to connect the probe in the side direction by providing the probe connector on the side of the body, a sub board for a connector may be required in addition to the main board. Accordingly, a structure of the body may become complex.

Accordingly, there is an increasing need for a simple structure where a probe connector with a probe may be provided on a main board without using a separate sub board for the probe connector.

In the conventional art, since the sub board is additionally provided in the body in addition to the main board, a number of electronic parts may increase, causing noise.

### SUMMARY

An aspect of the present invention provides a medical instrument that may directly form a probe connector on a main board without a need for a separate board in a movable type or a portable type of a body.

Another aspect of the present invention also provides a medical instrument that may reduce interference between a probe connector and a display unit. Here, the probe connector may include a diagnostic probe, and the display unit may display an inspection content of an inspection subject using an image.

Another aspect of the present invention also provides a medical instrument that may simplify a configuration of installing a movable type or a portable type body and a diagnostic probe and may reduce a time for mounting the diagnostic probe to the body.

Another aspect of the present invention also provides a medical instrument that may reduce electromagnetic waves occurring when connecting a body and a diagnostic probe, and may also reduce noise.

According to an aspect of the present invention, there is provided a medical instrument including: at least one diagnostic probe; a body having a main board embedded; a display unit being rotatably connected to one end of the body to display an inspection content of an inspection subject as an image; and a probe connector being provided on a top surface of the main board, which is externally exposed from the body, to be connected to the at least one diagnostic probe.

Through the above configuration, it is possible to form the probe connector using only the main board without using a separate board to form the probe connector. Also, it is possible to simplify a configuration of mounting the diagnostic probe to the body, and to reduce a time of mounting the diagnostic probe to the body.

Here, the probe connector may include a contact portion that may be electrically connected to the diagnostic probe. The contact portion may be formed in an upper direction or a side direction with respect to the main board. In this instance, the probe connector may be directly connected to the main board without using an intermediate medium such as a sub board. Since the probe connector is formed on a top surface of the main board, the diagnostic probe may be provided in the upper direction with respect to the body.

Also, an extended board with the probe connector may be provided on the body. The extended board may be integrally formed with the main board and be externally extended from the main board. Specifically, the extended board may be externally exposed from the body. Accordingly, when providing the probe connector on the extended board, the probe connector may be substantially connected to the main board. When the extended board is extended from the main board, the extended board and the main board may correspond to the same part. Therefore, the diagnostic probe connected to the extended board through the probe connector may be electrically connected to the main board of the body without using other devices.

Also, the extended board may be formed in a stepped shape or a groove shape with respect to the body. Specifically, the probe connector and the diagnostic probe may be provided on the extended board and thus may have a relatively lower height than the body. Through the above configuration, it is possible to reduce interference between the display unit and the probe connector or the diagnostic probe.

The extended board may be provided in at least one of a front portion, a rear portion, and a side portion of the body. Generally, the extended board is provided in the rear portion of the body and thereby is hidden by the body. Accordingly, it is possible to enhance an aesthetic aspect of the body. However, for a user necessity or for ease of use, the extended board may be provided in the front portion or the side portion of the body.

In particular, when the extended board is provided in the rear portion of the body, a hinge driving unit may be provided in a location where the extended portion starts, in order to prevent interference between the probe connector and the display unit.

Specifically, in order to prevent the display unit from contacting with the probe connector when opening the display unit on the body, the display unit may have a height greater than a height of the probe connector mounted onto the extended board. Accordingly, the hinge driving unit may make the display unit be opened on the top surface of the body, while the driving unit does not contact with the probe connector mounted onto the extended board.

Additional aspects, features, and/or advantages of the invention will be set forth in part in the description which follows and, in part, will be apparent from the description, or may be learned by practice of the invention.

### BRIEF DESCRIPTION OF THE DRAWINGS

These and/or other aspects, features, and advantages of the invention will become apparent and more readily appreciated from the following description of exemplary embodiments, taken in conjunction with the accompanying drawings of which:

FIG. 1 is a perspective view illustrating a medical instrument according to an embodiment of the present invention;

FIG. 2 is a perspective view illustrating a medical instrument including a probe connector according to an embodiment of the present invention;

FIG. 3 is a partially enlarged perspective view illustrating the probe connector of FIG. 2;

FIGS. 4A and 4B are cross-sectional views illustrating a cross section of a medical instrument according to an embodiment of the present invention;

FIG. 5 is a partially enlarged perspective view illustrating a probe connector according to an embodiment of the present invention; and

FIG. 6 is a perspective view illustrating a medical instrument according to another embodiment of the present invention.

### DETAILED DESCRIPTION

Reference will now be made in detail to exemplary embodiments of the present invention, examples of which are illustrated in the accompanying drawings, wherein like reference numerals refer to the like elements throughout. Exemplary embodiments are described below to explain the present invention by referring to the figures.

Prior to describing the present invention, a medical instrument according to an embodiment of the present invention may use a probe while a medical diagnostic process is being performed. For example, an ultrasonic diagnostic apparatus including a probe will be used herein for description, but the present invention is not limited thereto. Specifically, the present invention may be applicable to various types of medical instruments.

Also, a body mounted to the medical instrument may be used for a movable or portable medical instrument and may also be used for a fixed-type medical instrument that is placed in a certain place. Hereinafter, for ease of description, description will be made regarding a portable medical instrument that may be movable according to a user request, but the present invention is not limited thereto. The body may be a body that is mounted to a medical instrument fixed in a particular place.

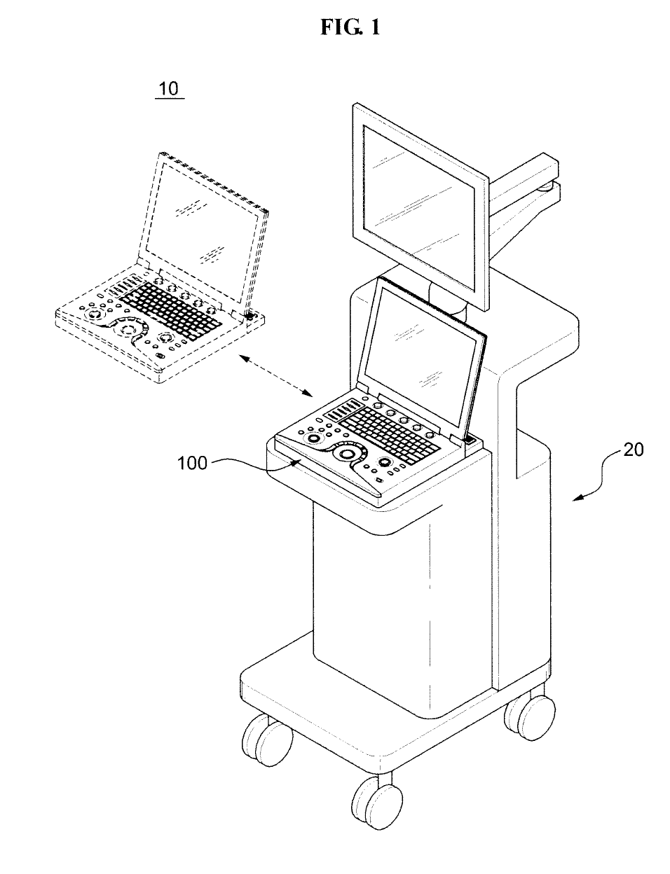

FIG. 1 is a perspective view illustrating a medical instrument 100 according to an embodiment of the present invention.

Referring to FIG. 1, an ultrasonic diagnostic apparatus 10 including the medical instrument 100 may include a diagnostic probe (not shown) and a storage device 20. the diagnostic probe may emit ultrasonic waves to an inspection subject and receive ultrasonic wave echoes from the inspection subject. The storage device may support the movable or portable medical instrument 100, may be connected to the medical instrument 100 to thereby support or fix the medical instrument 100, or may store ore output data transmitted from the medical instrument 100. In this instance, the diagnostic probe may be connected to the medical instrument 100 to inspect the inspection subject using ultrasonic waves. Depending on embodiments, the diagnostic probe may be connected to the storage device 20.

The medical instrument 100 may include a movable type or a portable type of a body that is detachable from the storage device 20. Accordingly, the medical instrument 100 may move to a location desired by the inspection subject to thereby perform inspection.

In order to perform an ultrasonic inspection for the inspection subject, the diagnostic probe may be required. During an inspection process, the diagnostic probe may be electrically connected to the medical instrument 100.

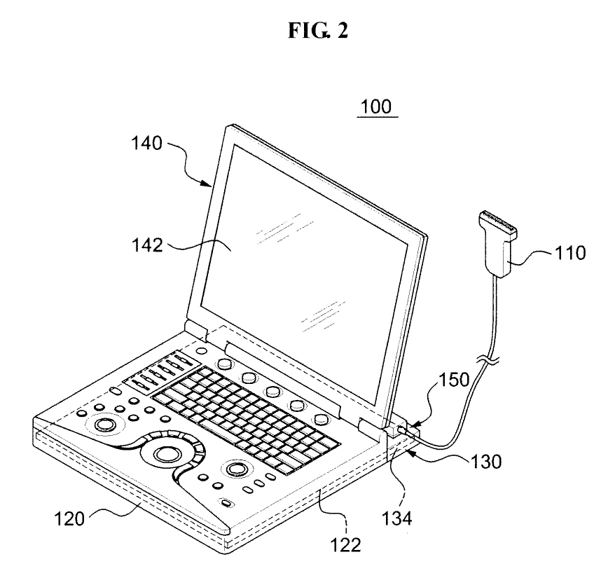

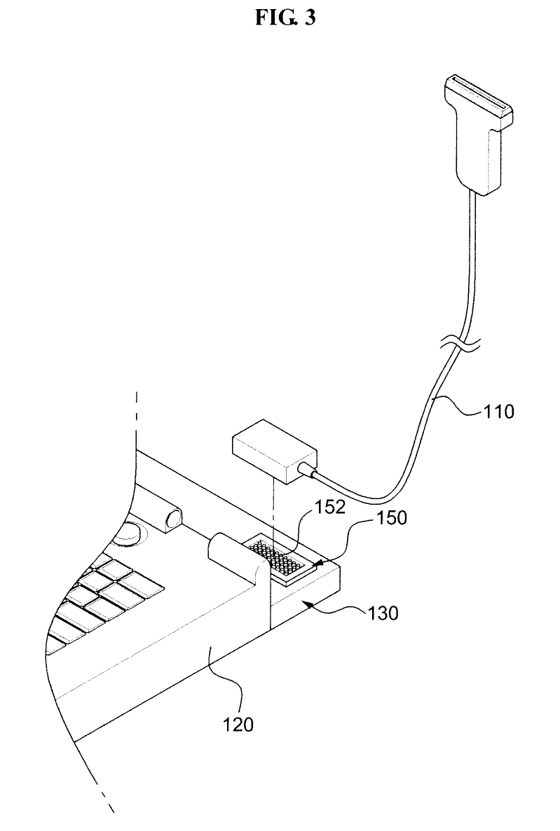

The above medical inspection using the diagnostic probe may need a probe connector (not shown) to electrically connect the diagnostic probe and the medical instrument 100. Hereinafter, the probe connector will be described in detail with reference to FIGS. 2 and 3.

FIG. 2 is a perspective view illustrating a medical instrument 100 including a probe connector 150 according to an embodiment of the present invention, and FIG. 3 is a partially enlarged perspective view illustrating the probe connector 150 of FIG. 2.

Referring to FIGS. 2 and 3, the medical instrument 100 may include a diagnostic probe 110, a body 120 having a main board 122 embedded, a display unit 140 being rotatably connected to one end of the body 120 to display an inspection content of an inspection subject using an image, and the probe connector 150 being provided on a top surface of the main board 122, which is externally exposed from the body 120, to be connected to the diagnostic probe 110.

Here, while a user is inspecting the inspection subject, the body 120 may include a control panel (not shown) that may store the inspection content in the body 120 or may control an operation of the diagnostic probe 110. The display unit 140 may include an image panel 142 that may display the inspection content using the image.

Here, the main board 122 may be provided in the body 120. The main board 122 may include a memory unit or various types of elements to store the inspection content of the inspection subject via the control panel. In this instance, the probe connector 150 to which the diagnostic probe 110 is connected may be provided on the outside of the body 120. In order to form the probe connector 150, an extended board 134 may be extended from the main board 122 to thereby be externally exposed from the body 120.

In particular, a housing 130 may be provided to prevent a contamination by foreign substances, a scratch, and the like. The housing 130 may be provided along an outer circumferential surface of the extended board 134 to protect the extended board 134. The probe connector 150 may be provided on the extended board 134.

The extended board 134 is integrally formed with the main board 122. Since one end of the main board 122 is externally exposed from the body 120, it can be said that the extended board 134 and the main board 122 have the same characteristic. Accordingly, when the probe connector 150 is formed on the extended board 134, the probe connector 150 may be electrically connected to the main board 122. Specifically, the diagnostic probe 110 connected to the probe connector 150 may be directly mounted to the main board 122.

The above extended board 134 may be provided in any one of a front portion, a rear portion, and a side portion of the body 120. Referring to FIGS. 2 and 3, the extended board 134 is provided in the rear portion of the body 120, but the present invention is not limited thereto. Specifically, for a user convenience, the extended board 134 may be provided in the front portion or the side portion of the body 120.

The probe connector 150 provided on the housing 130 may include a contact portion 152 that may receive the diagnostic probe 110. One end surface of the contact portion 152 may be connected to the extended board 134, and another end surface of the contact portion 152 may be connected to the diagnostic probe 110. Through this, when the diagnostic probe 110 is connected to the probe connector 150, the body 120 may be also electrically connected to the diagnostic probe 110.

Also, on the housing, the probe connector 150 may be formed in an upper direction or a side direction with respect to the body 120. Here, a case where the contact portion 152 is formed in the upper direction with respect to the body 120 will be described herein as an example.

When the probe connector 150 is provided on the top surface of the extended board 134 that is externally extended from the main board 122, and the contact portion 152 is formed in the upper direction with respect to the body 120, the diagnostic probe 110 may be provided along the upper direction of the body 120. Specifically, it is possible to mount the diagnostic probe 110 to the body 120 without using other devices.

In the conventional art, a diagnostic probe may be provided on the side of a body using a probe connector. In this case, no terminal that may be electrically connected to the body is provided on the side of the body. Accordingly, it is possible to connect the body and the probe connector by providing a sub board to connect the body and the probe connector. Specifically, an additional device may be used to connect the body and the diagnostic probe.

However, according to an embodiment of the present invention, it is possible to directly form the probe connector 150 on the extended board 134 that is externally extended from the main board 122. Accordingly, it is possible to connect the body 120 and the diagnostic probe 110 without using an additional device. Through this, a configuration of connecting the diagnostic probe 110 and the body 120 may be simplified. Also, a time for connecting the diagnostic probe 110 and the body 120 may be reduced.

Also, in a process of connecting the diagnostic probe 110 and the body 120, the diagnostic probe 110 may be mounted to the body 120 using a minimum configuration. Specifically, since other devices may be reduced between the diagnostic probe 110 and the body 120, it is possible to reduce electromagnetic waves caused by the other devices. Also, it is possible to reduce noise that may occur in the inspection process using the diagnostic probe 110.

Also, when the extended board 134 is provided in the rear portion of the body 120, it may be possible to enhance an aesthetic aspect of the body 120. Specifically, since the probe connector 150 with the probe 110 is hidden by the body 120 or the display unit 140, it is possible to enhance a beauty of an external appearance of the body 120.

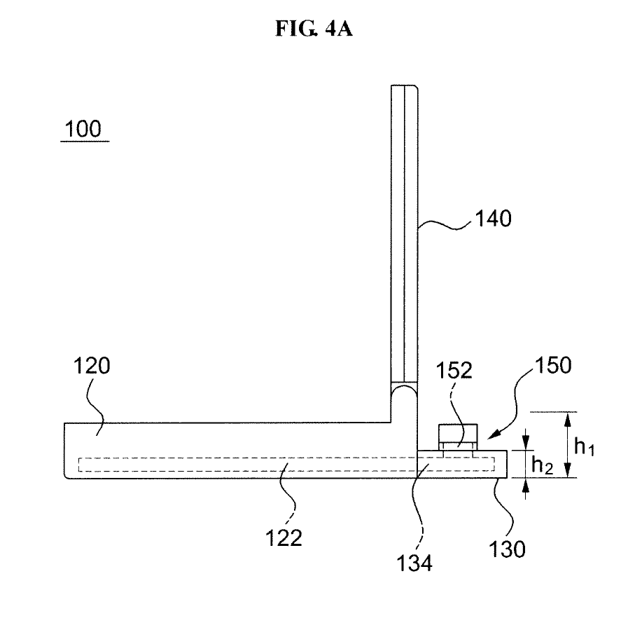

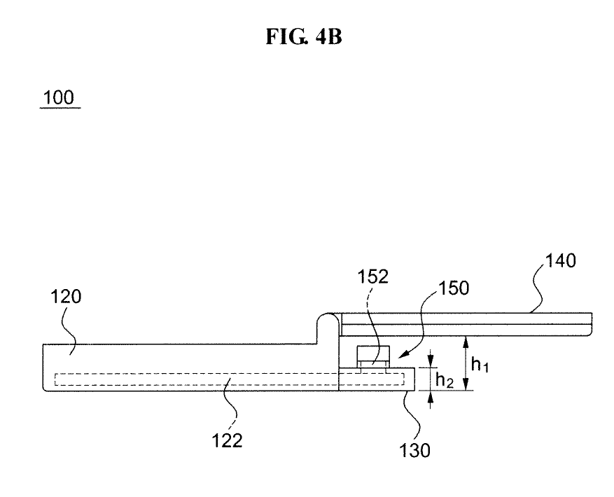

FIGS. 4A and 4B are cross-sectional views illustrating a cross section of a medical instrument 100 according to an embodiment of the present invention.

Referring to FIGS. 4A and 4B, a body 120 of the medical instrument 100 may include a display unit 140 that may display an inspection content of an inspection subject or other images. The display unit 140 may be hinge-rotatable on the body 120. For this, the display unit 140 may include a hinge driving unit (not shown).

According to an embodiment of the present invention, the hinge driving unit may be provided in a connection portion of the body 120 and the display unit 140. Specifically, the hinge driving unit may guide a hinge-rotation of the display unit 140. Also, when the display unit 140 is opened from the body 120, the hinge driving unit may adjust an angle of the display unit 140 to thereby prevent the display unit 140 from contacting with or colliding with the probe connector 150.

A connection portion of the body 120 and the extended board 134 may be formed in a stepped shape or a groove shape. In particular, a height h₂ of the extended board 134 may be less than or equal to a height h₁ of the body 120. Referring to figures, the height h₂ of the extended board 134 may be less than the height h₁ of the body 120 by forming the connection portion of the body 120 and the extended board 134 in the stepped shape or the groove shape. In a case where the height h₂ of the extended board 134 is less than the height h₁ of the body 120, although the user opens the display unit 140 from the body 120, it is possible to prevent interference from occurring between the display unit 140 and the probe connector 150 or a probe (not shown).

Referring to FIG. 4B, it is assumed that the display unit 140 is completely opened from the body 120 to a rear portion of the body 120. In this case, in order to prevent the display unit 140 from hanging over the probe connector 150 to thereby cause interference between the display unit 140 and the probe connector 150, it may be desirable to from the height h₂ of the extended board 134 to be less than the height h₁ of the body 120.

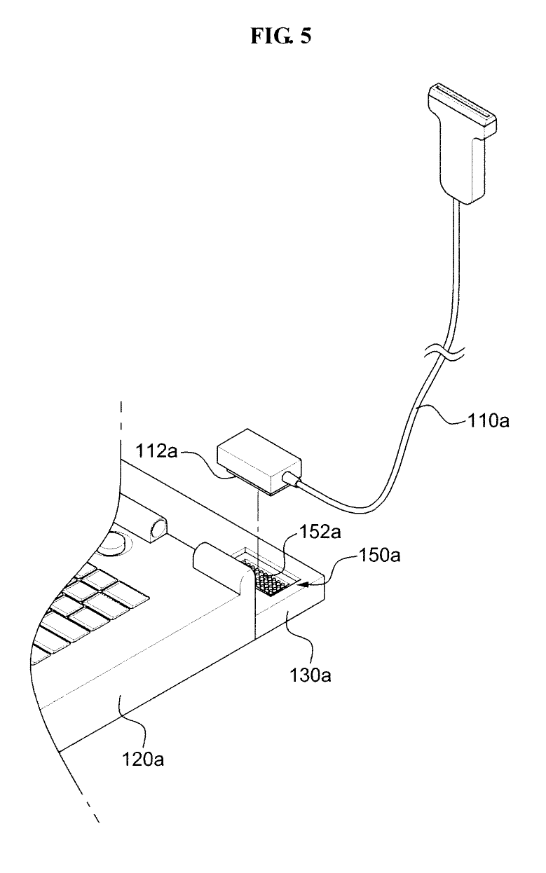

FIG. 5 is a partially enlarged perspective view illustrating a probe connector 150a according to an embodiment of the present invention.

Referring to FIG. 5, the probe connector 150a may be received in a housing 130a. Specifically, a contact portion 152a of the probe connector 150 that is connected to a diagnostic probe 110a may be provided in the housing 130a. Through this, when the probe connector 150a is disconnected from the diagnostic probe 110a, it is possible to protect the probe connector 150a against an external impact, or to prevent the probe connector 150a from being contaminated by foreign substances.

A connection portion 112a may be formed to connect the diagnostic probe 110a and the contact portion 152a of the probe connector 150a. The connection portion 112a may be extended from a bottom of the diagnostic probe 110a to have the same length as a depth that the probe connector 150a is received in the housing 130a.

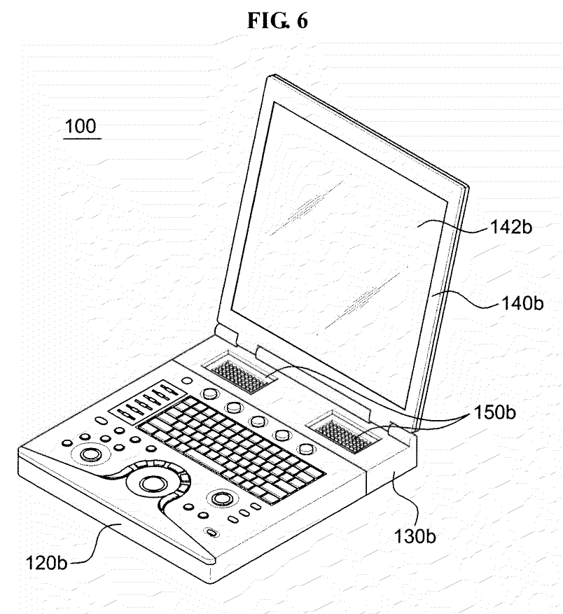

FIG. 6 is a perspective view illustrating a medical instrument 100 according to another embodiment of the present invention.

Referring to FIG. 6, the medical instrument 100 may include a body 120b, a housing 130b including a probe connector 150b that is received in a rear portion of the body 120b, and a display unit 140b including an image panel 142b that is connected to the body 120b to display an inspection content of an inspection object.

In a case where the probe connector 150b is received in the body 120b, although the display unit 140b covers the top surface of the body 120b after using a diagnostic probe (not shown), it is possible to prevent the probe connector 150b from being damaged due to the display unit 140 or external impact. Also, when the diagnostic probe is not used, the probe connector 150b may be included in the body 120b. Accordingly, it is possible to enhance an aesthetic aspect of the body 120b.

In this instance, after the display unit 140b covers the top surface of the body 120b, the probe connector 150b may be completely received in the housing 130b to thereby prevent interference between the display unit 140b and the probe connector 150b.

Although a few exemplary embodiments of the present invention have been shown and described, the present invention is not limited to the described exemplary embodiments. Instead, it would be appreciated by those skilled in the art that changes may be made to these exemplary embodiments without departing from the principles and spirit of the invention, the scope of which is defined by the claims and their equivalents.

What is claimed is:

1. A medical instrument comprising:
at least one diagnostic probe;
a body having a main board embedded;
a display unit being rotatably connected to one end of the body to display an inspection content of an inspection subject as an image; and
a probe connector being provided on a top surface of the main board, which is externally exposed from the body, to be connected to the at least one diagnostic probe.

2. The medical instrument of claim 1, wherein:
the probe connector includes a contact portion that is electrically connected to the main board and the at least one diagnostic probe, and
the contact portion is formed in an upper direction or a side direction with respect to the body.

3. The medical instrument of claim 2, wherein:
the body includes an extended board that is externally extended from the body, and
a housing is provided along an outer circumferential surface of the extended board to receive the extended board.

4. The medical instrument of claim 3, wherein the extended board is formed in a stepped shape or in a groove shape with respect to the body.

5. The medical instrument of claim 4, wherein the extended board is provided in at least one of a front portion, a rear portion, and a side portion of the body.

6. The medical instrument of claim 5, wherein the display unit is rotatably connected to the body in a location where the extended board starts, in order to prevent interference from occurring between the display unit and the extended board provided in the rear portion of the body.

7. The medical instrument of claim 3, wherein a height of the extended board is less than or equal to a height of the body.

8. The medical instrument of claim 1, wherein the probe connector is used for an ultrasonic diagnostic apparatus.

9. The medical instrument of claim 8, wherein the body is a movable type or a portable type.
